(19) United States
(12) Patent Application Publication
(10) Pub. No.: US 2011/0218362 A1
(43) Pub. Date: Sep. 8, 2011

(54) Fixed Bed Hydrogenation Of Fatty Nitriles To Fatty Amines

(75) Inventors: Daniel Ostgard, Kleinostheim (DE); Roberta Olindo, Munchen (DE); Virginie Duprez, Toulon (FR); Stefan Röder, Sinntal (DE); Monika Berweiler, Maintal (DE)

(73) Assignee: DEGUSSA GMBH, DUESSELDORF (DE)

(21) Appl. No.: 11/718,939

(22) PCT Filed: Nov. 10, 2004
(86) PCT No.: PCT/EP04/12694

§ 371 Date: Sep. 7, 2007

(51) Int. Cl. C07C 209/48

(52) U.S. Cl. 564/490

(57) ABSTRACT

A process for the fixed bed hydrogenation of unsaturated fatty nitriles to fatty amines with a fixed bed Raney-type Ni/Al, Co/Al or Ni/Co/Al catalyst in the liquid phase, the trickle phase or any type of fatty nitrile aerosol.

Activated metal catalysts are also known in the fields of chemistry and chemical engineering as Raney-type, sponge and/or skeletal catalysts. They are used, largely in powder form, for a large number of hydrogenation, dehydrogenation, isomerization and hydration reactions of organic compounds. These powdered catalysts are prepared from an alloy of a catalytically-active metal, also referred to herein as the catalyst metal, with a further alloying component which is soluble in alkalis. Mainly nickel, cobalt, copper or iron are used as catalyst metals. Aluminum is generally used as the alloying component which is soluble in alkalis, but other components may also be used, in particular zinc and silicon or mixtures of these either with or without aluminum.

These so-called Raney alloys are generally prepared by the ingot casting process. In that process a mixture of the catalyst metal and, for example, aluminum are first melted and casted into ingots. Typical alloy batches on a production scale amount to about ten to one hundred kg per ingot. According to DE 21 59 736 cooling times of up to two hours were obtained. This corresponds to an average rate of cooling of about 0.2 K/s. In contrast to this, rates of 102 to 106 K/s and higher are achieved in processes where rapid cooling is applied (for example an atomizing process). The rate of cooling is affected in particular by the particle size and the cooling medium (see Materials Science and Technology, edited by R. W. Chan, P. Haasen, E. J. Kramer, Vol. 15, Processing of Metals and Alloys, 1991, VCH-Verlag Weinheim, pages 57 to 110). A process of this type is used in EP 0 437 788 B 1 in order to prepare a Raney alloy powder. In that process the molten alloy at a temperature of 5 to 500° C. above its melting point is atomized and cooled using water and/or a gas. The invention of this patent can be applied to the catalysts prepared from slowly, moderately and rapidly cooled alloys. The use of cooling mediums, including but not limited to water, air and inert gases (e.g., Ar, He, N₂ and others) can also be used in fabricating the alloys, that are formed and activated with caustic solutions in order to generate the catalyst precursors used in this invention.

To prepare a powder catalyst, the Raney alloy is first finely milled, if it has not been produced in the desired powder form during preparation. Then the aluminum is partly (and if need be, totally) removed by extraction with alkalis such as, for example, caustic soda solution (other bases such as KOH are also suitable) to activate the alloy powder. Following extraction of the aluminum, the remaining catalytic power has a high specific surface area (BET), between 5 and 150 m²/g and is rich in active hydrogen. The activated catalyst powder is pyrophoric and stored under water or organic solvents or is embedded in organic compounds (e.g., distearyl amine), which are solid at room temperature.

These catalysts can also be promoted with one or more elements, coming from the periodic groups 1A, 2A, IIIB, IVB, VB, VIB, VIIB, VIII, IB, IIB, IIIA, IVA, VA and VIA. Preferably the promoting elements come from the periodic groups IIIB, IVB, VB, VIB, VIIB, VIII, IB, IIB, IIIA, IVA and VA. One or more of these promoting elements can be incorporated into the catalyst by either initially adding the element(s) to the precursor alloy before leaching or by adsorbing the element(s) either during or after the activation of the catalyst. A combination of promotion methods could also be used as one or more promoting elements are given to the precursor alloy, and the others, or in some cases more of the same element(s) are adsorbed onto the catalyst as it is being activated, after it has been activated and washed or a combination of both.

The powdered activated base metal catalysts (Raney-type catalysts) are typically used in batchwise processes with stirred tank reactors. For the production of lower quantities of product, these batchwise processes are very flexible and economically feasible. Nonetheless, the constant cycle of startup, reactor charging, heating the reactor, performing the reaction, cooling the reactor, reactor discharging, the separation of the catalyst from the reaction mixture and catalyst recycle to the next batch make this process very complicated and labor intensive. Moreover, this sequence of complicated labor intensive steps provides the operators of this process with many opportunities and sources of error that could have serious safety and economical consequences. In this respect, the use of continuous reaction technology provides for a less labor intensive process that has fewer sources of error. Additionally, if one wants to produce larger quanties of products, this could be performed under better economical conditions with one of the continuous processes mentioned in this patent. It is known to those skilled in the art (see: J. Super, in “Catalysis of Organic Reactions”, Michael Ford editor, Marcel Dekker Inc., New York (2000) 35.), that a continuous process becomes more profitable than one carried out batchwise as the amount of product needed to be produced increases. The markets for both unsaturated and saturated fatty amines are large enough, so that the practitioner of a continuous production technology for these products would clearly enjoy an economic advantage over the current batchwise state of the art practiced broadly in this industry. Continuous processes are best carried out with fixed bed catalysts, where the problems involved with catalyst separation from the reaction mixture are readily solved without the use of additional equipment and the use of labor intensive procedures similar to those mentioned above. Through the use of a continuous reaction technology with a fixed bed catalyst, it will be possible to not only control the rate of production, but also the product selectivity, where slower throughputs and the corresponding longer residence times of fatty nitrile and its intermediate products in the catalyst bed lead to higher percentages of saturated amines. Logically, faster throughputs with shorter residence times in the catalyst bed lead to a more selective hydrogenation of the stronger adsorbed nitrile part of the unsaturated fatty nitrites to afford a higher selectivity of unsaturated fatty amines with higher iodine value retentions, meaning that the conversion of the olefin functionality is very low. While changing the operational parameters of a continuous process with a fixed bed catalyst can provide one with a high level of flexibility in product selectivity, activity and lifetime, additional flexibility can be provided through the design of the fixed bed catalyst, where parameters such as the level of activation, the type of activation procedure, the size and shape of the fixed bed catalyst, the type and number of catalytic metals in the catalyst, the presence of promoters and the number of promoters can all play an important role in the design of the best catalyst for the desired product distribution.

Examples of the fixed bed forms of activated base metal catalysts used in this invention include, but are not limited to, tablets (Schutz et al. EP648535, Freund et al. DE19721898, Ostgard et al. U.S. Pat. No. 6,489,521, Ostgard et al. U.S. Pat. No. 6,284,703), extrudates (Sauer et al. EP0880996) and Cheng et al. U.S. Pat. No. 4,826,799), activated hollow spheres (Ostgard et al. DE10101647, Ostgard et al. DE10101646, Ostgard et al. DE10065031, Ostgard et al. U.S. Pat. No. 6,486,366, Ostgard et al. U.S. Pat. No. 6,437,186, Ostgard et al. EP1068900), activated flakes or fiber forms (e.g., tablets and mats in Ostgard et al. EP1068896), granules (formed by the agglomeration of alloy powders with binders and pore builders), supported activated catalytic metal/Al alloys, activated Al treated catalytic metal sheets and activated Al treated monliths containing a catalytic metal, that can alloy with the Al and can be activated with caustic to the catalyst. Raney-type fixed bed catalysts can also be made by the leaching (e.g., via caustic activation and its variations, vide-supra) of chunks of alloy, consisting of base metals with optionally one or more promoters and alkali leachable metals such as Al, Zn, Si or combinations thereof. The precursor alloy chunks can be formed by coarsed grinding of casted slowly cooled alloys, the controlled solidification of gas (e.g., nitrogen or air) cooled alloys, the controlled solidification of liquid (e.g., water) cooled alloys or the controlled solidification of gas and liquid cooled alloys. An example of such a controlled cooling processe include the cooling of the alloy, melt to about 5 to 200° C. or preferably 10 to 100° C., above the solidification temperature before introducing it into the liquid or gas cooling medium. The chunks can then be formed by either adding the cooled melt to the cooling medium (e.g., water) dropwise, where the size of the drops and the corresponding chunks are controlled via the opening of the dripping device or in a continuous stream, that may be interrupted mechanically before the alloy is quenched. The final, initial or combined cooling rates of these chunks of alloy may vary from 0.2 to 106 K/s via the methods mentioned above. The above mentioned chunks of alloy may be actived by causticly (or by the use of other bases as well) leaching away the desired amount of Al, as was mentioned previously for the powdered catalysts.

Fixed bed catalysts are also optionally promoted with one or more elements from the periodic groups 1A, 2A, IIIB, IVB, VB, VIB, VIIB, VIII, IB, IIB, IIIA, IVA, VA, VIA and the rare earth elements. Preferably the promoting elements come from the periodic groups IIIB, IVB, VB, VIB, VIIB, VIII, IB, IIB, IIIA, IVA, VA and the rare earth elements. One or more of these promoting elements can be incorporated into the catalyst by either initially adding the element(s) to the precursor alloy before leaching or by adsorbing the element(s) either during or after the activation of the catalyst. Promotion with combinations of the above mentioned elements can also be accomplished by using a combination of techniques, where one or more element(s) are added into the alloy and the other(s) or more of the same are/is added during or after leaching the alloy with caustic solutions.

The present invention relates to the use of the above described fixed bed activated base metal catalysts for the improved hydrogenation of fatty nitrites to their corresponding fatty amines via a continuous process.

The subject of the invention is a process for the fixed bed hydrogenation of unsaturated fatty nitrites with a fixed bed Raney-type Ni/Al, Co/Al or Ni/Co/Al catalyst in the liquid phase, the trickle phase or any type of fatty nitrile aerosol.

In the process for the fixed bed hydrogenation of unsaturated fatty nitrites with a fixed bed Raney-type Ni/Al, Co/Al or Ni/Co/Al catalyst in the liquid phase, the trickle phase or any type of fatty nitrile aerosol according to the invention, the catalyst can be doped with one or more of the elements from the group of Mo, Fe, Cr, Co, Cu or Ni.

In the process for the fixed bed hydrogenation of unsaturated fatty nitrites with a fixed bed Raney-type Ni/Al catalyst in the liquid phase, the trickle phase or any type of fatty nitrile aerosol according to the invention, the catalyst can be doped with one or more of the elements from the group of Mo, Fe, Cr, Cu or Co.

In the process for the fixed bed hydrogenation of unsaturated fatty nitrites with a fixed bed Raney-type Co/Al catalyst in the liquid phase, the trickle phase or any type of fatty nitrile aerosol according to the invention, the catalyst can be doped with one or more of the elements from the group of Mo, Fe, Cr, Cu or Ni.

In the process for the fixed bed hydrogenation of unsaturated fatty nitrites with a fixed bed Raney-type Co/Al catalyst in the liquid phase, the trickle phase or any type of fatty nitrile aerosol where according to the invention, the catalyst can be doped with one or more of the elements from the group of Mo, Fe, Cr, Cu or Ni and treated with LiOH.

In the process for the fixed bed hydrogenation of unsaturated fatty nitrites with a fixed bed Raney-type Ni/Al, Co/Al or Ni/Co/Al catalyst in the liquid phase, the trickle phase or any type of fatty nitrile aerosol according to the invention, the catalyst can be doped with one or more of the elements from the periodic table group of 1A, 2A, IIIB, IVB, VB, VIB, VIIB, VIII, IB, IIB, IIIA, IVA, VA, VIA and the rare earth elements.

In the process for the fixed bed hydrogenation of unsaturated fatty nitrites with a fixed bed Raney-type Ni/Al, Co/Al or Ni/Co/Al catalyst in the liquid phase, the trickle phase or any type of fatty nitrile aerosol according to the invention, the catalyst can be doped with one or more of the elements from the periodic table group of IIIB, IVB, VB, VIB, VIIB, VIII, IB, IIB, IIIA, IVA, VA and the rare earth elements.

In the process for the fixed bed hydrogenation of unsaturated fatty nitrites according to the invention, the feed can pass only one time through the catalyst bed.

In the process for the fixed bed hydrogenation of unsaturated fatty nitrites according to the invention, the feed can be recycled continuously through the catalyst bed until the desired product is made.

In the process for the fixed bed hydrogenation of unsaturated fatty nitrites according to the invention, the product and/or solvent can be recycled continuously through the catalyst bed and only enough of the feed can be added to the recycled stream that can be reacted via one pass and the amount of product removed after the catalyst bed is equal to the amount of feed added before it.

In the process for the fixed bed hydrogenation of unsaturated fatty nitrites according to the invention, the feed can be sent through a series of reactors and the conversion of the feed increases as it passed through more reactors.

In the process for the fixed bed hydrogenation of unsaturated fatty nitrites according to the invention, the hydrogenation can be carried out at pressures ranging from 20 to 100 bars and temperatures from 80 to 160° C.

In the process for the fixed bed hydrogenation of unsaturated fatty nitrites according to the invention, the hydrogenation can be carried out at pressures ranging from 1 to 300 bars and temperatures from 50 to 200° C.

In the process for the fixed bed hydrogenation of unsaturated fatty nitrites according to the invention, the hydrogenation can be carried out in the presence of one or more bases.

In the process for the fixed bed hydrogenation of unsaturated fatty nitrites according to the invention, the hydrogenation can be carried out in the presence of ammonia.

In the process for the fixed bed hydrogenation saturated fatty nitrites can be hydrogenated to saturated fatty amines.

In the process for the fixed bed hydrogenation saturated fatty nitrites can be hydrogenated to primary saturated fatty amines.

In the process for the fixed bed hydrogenation whereby triglycerides can be hydrogenated.

In the process for the fixed bed hydrogenation unsaturated fatty nitrites can be hydrogenated to primary unsaturated fatty amines.

In the process for the fixed bed hydrogenation unsaturated fatty nitrites can be hydrogenated to primary saturated fatty amines.

The fatty nitrites can be saturated or unsaturated fatty nitrites. The fatty amines encompassed in this invention are straight-chain primary, secondary and tertiary amines with chain lengths between 6 and 24 carbon atoms, containing from 3 to 0 olefinic double bonds per aliphatic chain, that can be prepared via the hydrogenation of their precursor fatty nitrites.

Some of the commercially interesting fatty amines and their natural mixtures, produced by this invention include, but are not limited to, oleyl amines, stearyl amines, linoleyl amines, myristyl amines, palmityl amines, lauryl amines, cocoyl amines, tallow amines, saturated tallow amines and soya amines, as well as, those fatty amine mixtures resulting from the conversion of tall oils, cottonseed oil, grapeseed oil, ground nut oils, lards, linseed oil, corn oil, olive oil, rapeseed oil, rice bran oil, safflower oil, sesame oil, sunflower oil, teaseed oil, tomatoseed oil, marine oils (e.g., fish, seal and sea elephant oils), castor oil and mixtures thereof to name a few.

Fatty amines are generally produced by the hydrogenation of fatty nitrites, that originate from the conversion of naturally occuring fats and oils to the corresponding fatty acids and glycerol, followed by the conversion of the resulting fatty acids with ammonia at ˜280-360° C. and atmospheric pressure over bauxite, ZnO, Mn or Co catalysts to the desired fatty nitrites (S. Billenstein, G. Blaschke, JAOCS, vol. 61, no 2 (1984), 353).

It is well known that the catalytic hydrogenation of nitrites may produce a mixture of primary, secondary and tertiary amines, as first proposed by Von Braun et al. in 1923 (J. Von Braun, G. Blessing and F. Zobel, Chem. Ber., 56 (1923) 1988).

The selectivity of this reaction to primary amines can be improved by the addition of bases, including but not limited to NaOH, KOH, LiOH and ammonia. One of the main product groups of this invention is that of primary fatty amines, that are used as flotation reagents, corrosion inhibitors, asphalt emulsifiers, chemical intermediates to other surfactants and numerous other applications. As previously discussed, these fatty amines may either be saturated or unsaturated as determined by the desired properties of the end product, and it is the catalyst together with the hydrogenation procedure, that will determine if the end product is saturated, unsaturated or unsaturated to a desired level, and if it is a primary, secondary, tertiary or a combination of 2 or 3 of these amines.

The process of this invention may be carried out with hydrogen percolated into and dissolved into the feed from either the top or the bottom of the reactor, with hydrogen percolated into and dissolved into the feed from different entry points along the length of the reactor, with hydrogen percolated into and dissolved into the feed in a direction countercurrent to the feed or with hydrogen percolated into and dissolved into the feed in the same direction as the feed's current. The process of this invention can be carried out in a fixed bed reactor via the trickle phase, the liquid phase in a flooded fixed bed reactor and with any kind of aerosol of the fatty nitrile. This process can be carried out either with or without the use of a solvent.

The hydrogenation of this invention can be carried out so, that the complete conversion occurs under such conditions that the fatty nitrile only needs to pass through the fixed bed reactor once.

This invention also incompasses the recirculation of the feed through the fixed bed reactor so, that its level of reduction is increased with each and every pass through the fixed bed reactor for a desired amount of passes to reach the desired product. Another recycling process encompassed in this invention is, where only enough of the fatty nitrile is added to the recycling product and/or solvent such, that it is immediately during one pass hydrogenated and the amount of product removed from the reactor, after the fixed catalyst bed is equivalent to the amount of reactant added before the fixed catalyst bed.

The recycling procedures of this invention can be carried out in a traditional tube reactor with a recirculation loop or in loop reactors (e.g., Buss Loop Reactors) and varieties of reactors, based on the principles of this reactor type, where the fixed bed catalyst is placed in the reaction zone of the reactor.

The hydrogenation of this invention can also be carried out through a series of reactors, where the first reactor brings the conversion of the fatty nitrile to a certain level of the desired fatty amine, and the reactors, that follow, increase the conversion level even further until the desired fatty amine at the desired conversion level is reached with the last reactor.

In this operation, the last one or two reactor(s) will be operating somewhat like polishing reactors and as such, the catalysts in the last reactors may last longer. In this case, one could change out the initial reactors more frequently than the later ones, due to their higher hydrogenation workload and keep the start of the reaction at the same reactor of this series of reactors.

One may also rotate the starting point of the reaction to the next reactor in the series as the old starting reactor is being changed out.

It is usually preferred, that only one reactor is changed out at a time, so that the reaction can continue with the other reactors during this change out. However this does not need to be the case.

In such a rotation scheme, the newly changed out reactor could be used as the last reactor in the series, where the end product is being polished. In this system, as the reactors are changed out in sequence, each reactor will find itself, sooner or later, at the start of the reaction towards the end of its catalyst charge's lifetime.

Another variety would be to make the new changed out reactor the starting point and as the containing catalyst charge ages during the sequential reactor change outs. This reactor will eventuall, become the polishing reactor at the end of this series.

This invention can also be reduced to practice via the outfitting of traditional stirred tank reactors with the appropriate catalyst basket technology, so that the above mentioned single pass, or in other words, single batch process from fatty nitrile to desired fatty amine and the recycling processes of this invention can also be carried out with one or more stirred tank reactors as dependent on the chosen process. The catalyst basket could be stationary, where the stirrer of the tank reactor forces the reaction mixture through the catalyst bed, or the catalyst basket could be a part of the stirrer itself, where the catalyst bed is swept through the reaction mixture.

This invention can also be applied towards the fixed bed hydrogenation of triglycerides, where their olefin moieties, as monitored by the molecule's iodine value, are hydrogenated to provide either totally saturated triglycerides or triglycerides of a certain level of unsaturation as determined upon the hydrogenation process, the design of the catalyst and the reaction conditions such as the LHSV, temperature and hydrogen pressure.

The above and other objects of the invention are achieved by the hydrogenation of fatty nitrites via a continuous process over a fixed bed catalyst with either a single pass, multiple pass or recirculating process. This hydrogenation can be carried our with either one fixed bed reactor, a series of fixed bed reactors, a loop reactor (e.g., a Buss loop reactor), one converted stirred tank reactor, where a stationary catalyst basket is built in, a series of converted stirred tank reactor, where a stationary catalyst basket is built in, one converted stirred tank reactor, where the catalyst basket is a part of the stirrer and/or series of converted stirred tank reactor, where the catalyst basket is a part of the stirrer.

This hydrogenation can be carried out in the liquid phase, the trickle phase and/or with any type of aerosol of the fatty nitrile, and this may be performed in either the presence or the absence of a solvent.

This invention can be used to hydrogenate one or more straight-chain primary, secondary and tertiary fatty nitrites with chain lengths between 6 and 24 carbon atoms containing from 3 to 0 olefinic double bonds per aliphatic chain. The most common feeds are those having one or more straight-chain primary fatty nitrites with chain lengths between 6 and 24 carbon atoms, containing from 3 to 0 olefinic double bonds per aliphatic chain.

This process can be optimized to yield the desired product from the correspondingly available feed leading to a satisfactory activity and catalyst lifetime to make this process commercially attractive. These optimization parameters include the reaction conditions and throughput, as well as the design of the catalyst itself.

The improvements in selectivity can involve enhanced chemo- and regioselective transformations to provide fatty amines with very high iodine value retentions at the desired levels of primary, secondary or tertiary amines.

Another option of this invention is the production of saturated fatty amines via the complete hydrogenation of the feed to the wished levels of primary, secondary and tertiary amines.

It is also possible to produce a desired mixture of primary, secondary and tertiary amines with the chosen level of saturation, that is lower than that of the initial fatty nitrile but not to completion.

The most sought after products tend to be primary unsaturated fatty amines and primary saturated fatty amines. The selectivity of this reaction to primary amines can be improved by the addition of bases including but not limited to NaOH, KOH, LiOH and ammonia.

The catalysts that can be used with this invention are fixed bed Raney-type Ni/Al, Ni/Mo/Al,Co/Al, Fe/Al, Co/Ni/Al, Co/Ni/Fe/Al and other commonly known varieties of these catalysts (such as those containing Cu and other metals), that may or may not be doped with one or more elements from the periodic groups 1A, 2A, IIIB, IVB, VB, VIB, VIIB, VIII, IB, IIB, IIIA, IVA, VA, VIA and the rare earth elements.

The common fixed bed forms included in this invention are extrudates, tablets, granules, activated chunks where the original alloy was solidified in a controlled way (slowly, rapidly and/or combinations thereof), hollow spheres, hollow spheres with different layers of different elements, hollow extrudates, fiber/flake tablets/mats, monoliths, metal sheets and supported Raney-type catalysts.

The catalysts can be used in the slurry phase, trickle phase, gas phase and/or combinations thereof. This invention also applies towards the fixed bed hydrogenation of triglycerides.

### EXAMPLE 1

### Production of Activated Raney-Type Ni Hollow Spheres

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the 53 wt.-% Ni/47 wt.-% Al alloy and Ni binder onto a fluidized bed of styrofoam balls (polystyrene balls). This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH˜10.5) before use. The final catalyst had a bulk density of 0.97 g/ml.

### EXAMPLE 2

### Production of Mo Doped Activated Raney-Type Ni Hollow Spheres

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of a Ni/Mo/Al alloy (˜50% Al) and Ni binder onto a fluidized bed of styrofoam balls (polystyrene balls). This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH ˜10.5) before use. The final catalyst had a bulk density of 1.00 g/ml.

### EXAMPLE 3

### Production of Activated Raney-Type Co Hollow Spheres

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the 50 wt. % Co/50 wt.-% Al alloy onto a fluidized bed of styrofoam balls (polystyrene balls). This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH ˜10.5) before use. The final catalyst had a bulk density of 0.93 g/ml.

### EXAMPLE 4

### Production of Cr/Ni Doped Activated Raney-Type Co Hollow Spheres

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of a Co/Ni/Cr/Al alloy (˜50% Al) onto a fluidized bed of styrofoam balls (polystyrene balls). This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH ˜10.5) before uses. The final catalyst had a bulk density of 0.85 g/ml.

### EXAMPLE 5

### Production of LiOH Treated Cr/Ni Doped Activated Raney-Type Co Hollow Spheres

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of a Co/Ni/Cr/Al alloy (˜50% Al) onto a fluidized bed of styrofoam balls (polystyrene balls). This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH ˜10.5) before being treated with LiOH. The LiOH treatment was carried out by dipping a basket with 100 ml of the precursor catalyst into a stirred beaker containing 400 grams of an aqueous 10 wt. % LIOH solution for one hour at room temperature. At the end of the hour, the catalyst basket was removed and dipped into a stirred beaker containing 400 ml of distilled water. This washing procedured was repeated two more times before the catalyst was stored under water. The final catalyst had a bulk density of 0.83 g/ml.

### APPLICATION EXAMPLE 1

### The Fixed Bed Hydrogenation of a Tallow Nitrile Mixture with Fixed Bed Raney-Type Activated Base Metal Catalysts

The fixed bed hydrogenation of a tallow nitrile mixture consisting predominantly of C₁₆ and C₁₈ with a small amount of C₁₄, C₂₀ and other long chain aliphatic fatty nitrites having an overall iodine value (IV) of ˜51 was carried out with a tube reactor in the trickle phase over 60 ml of catalyst at the pressure of 60 bars with a fourfold excess of hydrogen with respect to the total saturation of the tallow nitrile mixture. The reaction was carried out with the temperature sequence of 140, 110 and occasionally 90° C. where the LHSV (liquid hourly space velocity) sequence of 3, 2, 1 and 0.5 h-1 was used at each temperature. Two or three samples were collected for every LHSV. The testing of each temperature with four LHSV required one day and between test days, the catalyst was washed with a flow of 2 ml of ethanol per minute for 30 minutes as the reactor cooled down under 60 bars of hydrogen flowing at 22 liters per hour. After the initial 30 minute wash the ethanol flow was reduced to 0.1 ml per minute under 30 bars of hydrogen flowing at 22 liters per hour while the catalyst cooled down the rest of the way to room temperature and stayed that way until the next morning, when the tallow nitrile hydrogenation test started with the next reaction temperature.

The iodine value (IV), secondary and tertiary amine value (2/3A) and the total amine value (TAV) were all determined for the fresh tallow nitrile and the hydrogenation samples.

The IV was determined by a modified Wijs method similar to method Tg 1-64 of the American Oil Chemists' Society (AOCS), where the only difference was the use of cyclohexane instead of carbon tetrachloride. The 2/3A value was determined by the official AOCS method Tf 2a-64 and the TAV was measured via the AOCS potenziometric titration method Tf 1a-64.

These results are listed in Table 1.

TABLE 1
The results of the fixed bed hydrogenation of a
tallow nitrile mixture.

| | | | Total | Secondary and | |
|---|---|---|---|---|---|
| | Temperature | | Amine | Tertiary | Iodine |
| Catalyst | ° C. | LHSV h-1 | Value | Amine Value | Value |
| El | 140 | 3 | 93.3 | 25.4 | 40.7 |
| | | 2 | 116.4 | 33.1 | 33.4 |
| | | 1 | 149.5 | 46.8 | 20.1 |
| | | 0.5 | 161.0 | 56.5 | 9.5 |
| | 110 | 3 | 49.9 | 12.3 | 47.9 |
| | | 2 | 59.9 | 15.6 | 45.7 |
| | | 1 | 88.6 | 24.2 | 38.2 |
| | | 0.5 | 127.6 | 37.1 | 26.7 |
| E2 | 140 | 3 | 105.9 | 26.1 | 29.0 |
| | | 2 | 119.6 | 33.3 | 23.8 |
| | | 1 | 140.3 | 47.1 | 14.1 |
| | | 0.5 | 149.3 | 62.1 | 8.2 |
| | 110 | 3 | 59.3 | 11.8 | 40.4 |
| | | 2 | 78.9 | 16.2 | 35.5 |
| | | 1 | 102.5 | 22.5 | 27.2 |
| | | 0.5 | 134.9 | 31.2 | 18.9 |
| | 90 | 3 | 28.7 | 7.2 | 45.7 |
| | | 2 | 45.0 | 9.7 | 42.6 |
| | | 1 | 69.5 | 14.4 | 37.9 |
| | | 0.5 | 86.8 | 23.0 | 28.6 |
| E3 | 140 | 3 | 76.6 | 9.0 | 50.4 |
| | | 2 | 97.5 | 12.4 | 45.9 |
| | | 1 | 136.7 | 19.8 | 33.7 |
| | | 0.5 | 170.7 | 27.7 | 19.3 |
| E4 | 140 | 3 | 95.1 | 6.3 | 44.5 |
| | | 2 | 119.8 | 6.9 | 40.0 |
| | | 1 | 162.8 | 10.4 | 27.8 |
| | | 0.5 | 192.6 | 15.6 | 14.0 |
| | 110 | 3 | 76.2 | nd | 48.9 |
| | | 2 | 106.0 | 3.1 | 46.3 |
| | | 1 | 149.1 | 5.4 | 37.5 |
| | | 0.5 | 181.8 | 7.9 | 24.7 |
| E5 | 140 | 2 | 141.1 | 9.8 | 39.1 |
| | | 1 | 176.2 | 9.8 | 26.1 |
| | | 0.5 | 202.4 | 11.9 | 12.4 |

1. A process for the fixed bed hydrogenation of fatty nitrites with a fixed bed Raney-type Ni/Al, Co/Al or Ni/Co/Al catalyst in the liquid phase, the trickle phase or any type of fatty nitrile aerosol.

2. A process for the fixed bed hydrogenation of fatty nitrites with a fixed bed Raney-type Ni/Al, Co/Al or Ni/Co/Al catalyst in the liquid phase, the trickle phase or any type of fatty nitrile aerosol according to claim 1, where the catalyst is doped with one or more of the elements from the group of Mo, Fe, Cr, Co, Cu or Ni.

3. A process for the fixed bed hydrogenation of fatty nitrites with a fixed bed Raney-type Ni/Al catalyst in the liquid phase, the trickle phase or any type of fatty nitrile aerosol according to claim 1, where the catalyst is doped with one or more of the elements from the group of Mo, Fe, Cr, Cu or Co.

4. A process for the fixed bed hydrogenation of fatty nitrites with a fixed bed Raney-type Co/Al catalyst in the liquid phase, the trickle phase or any type of fatty nitrile aerosol according to claim 1, where the catalyst is doped with one or more of the elements from the group of Mo, Fe, Cr, Cu or Ni.

5. A process for the fixed bed hydrogenation of fatty nitrites with a fixed bed Raney-type Co/Al catalyst in the liquid phase, the trickle phase or any type of fatty nitrile aerosol where according to claim 1, the catalyst is doped with one or more of the elements from the group of Mo, Fe, Cr, Cu or Ni and treated with LiOH.

6. A process for the fixed bed hydrogenation of fatty nitriles with a fixed bed Raney-type Ni/Al, Co/Al or Ni/Co/Al catalyst in the liquid phase, the trickle phase or any type of fatty nitrile aerosol according to claim 1, where the catalyst is doped with one or more of the elements from the periodic table group of 1A, 2A, IIIB, IVB, VB, VIB, VIIB, VIII, IB, IIB, IIIA, IVA, VA, VIA and the rare earth elements.

7. A process for the fixed bed hydrogenation of fatty nitrites with a fixed bed Raney-type Ni/Al, Co/Al or Ni/Co/Al catalyst in the liquid phase, the trickle phase or any type of fatty nitrile aerosol according to claim 1, where the catalyst is doped with one or more of the elements from the periodic table group of IIIB, IVB, VB, VIB, VIIB, VIII, IB, IIB, IIIA, IVA, VA and the rare earth elements.

8. A process for the fixed bed hydrogenation of fatty nitrites according to claim 1, where the feed passes only one time through the catalyst bed.

9. A process for the fixed bed hydrogenation of fatty nitrites according to claim 1, where the feed is recycled continuously through the catalyst bed until the desired product is made.

10. A process for the fixed bed hydrogenation of fatty nitrites according to claim 1, where the product and/or solvent is recycled continuously through the catalyst bed and only enough of the feed is added to the recycled stream that can be reacted via one pass and the amount of product removed after the catalyst bed is equal to the amount of feed added before it.

11. A process for the fixed bed hydrogenation of fatty nitrites according to claim 1, where the feed is sent through a series of reactors and the conversion of the feed increases as it passed through more reactors.

12. A process for the fixed bed hydrogenation of fatty nitrites according to claim 1, where the hydrogenation is carried out at pressures ranging from 20 to 100 bars and temperatures from 80 to 160° C.

13. A process for the fixed bed hydrogenation of fatty nitrites according to claim 1, where the hydrogenation is carried out at pressures ranging from 1 to 300 bars and temperatures from 50 to 200° C.

14. A process for the fixed bed hydrogenation of fatty nitrites according to claim 1, where the hydrogenation is carried out in the presence of one or more bases.

15. A process for the fixed bed hydrogenation of fatty nitriles according to claim 1, where the hydrogenation is carried out in the presence of ammonia.

16. A process for the fixed bed hydrogenation of fatty nitrites according to claim 1, whereby saturated fatty nitrites are hydrogenated to saturated fatty amines.

17. A process for the fixed bed hydrogenation of fatty nitriles according to claim 1, whereby saturated fatty nitrites are hydrogenated to primary saturated fatty amines.

18. A process for the fixed bed hydrogenation according to claim 1, whereby triglycerides are hydrogenated instead of fatty nitrites.

19. A process for the fixed bed hydrogenation of fatty nitrites according to claim 1, whereby unsaturated fatty nitrites are hydrogenated to saturated fatty amines.

20. A process for the fixed bed hydrogenation of fatty nitriles according to claim 1, whereby unsaturated fatty nitrites are hydrogenated to saturated fatty amines.
